(19) United States
(12) Patent Application Publication
(10) Pub. No.: US 2016/0195502 A1
(43) Pub. Date: Jul. 7, 2016

(54) METHOD FOR CHIRAL SEPARATION OF METHAMPHETAMINE AND AMPHATAMINE ENANTIOMERS

(71) Applicant: Castle Medical, LLC, Smyrna, GA (US)

(72) Inventors: Sravan Kumar Mansani, Atlanta, GA (US); Thomas David, Atlanta, GA (US)

(21) Appl. No.: 14/985,771

(22) Filed: Dec. 31, 2015

Related U.S. Application Data
(60) Provisional application No. 62/098,820, filed on Dec. 31, 2014.

(51) Int. Cl. G01N 30/72; G01N 30/06; G01N 33/487

(52) U.S. Cl. CPC G01N 30/7266; G01N 33/487; G01N 30/06

(57) ABSTRACT

Methods for routine high throughput analysis with accurate results to determine the concentration of d- and/or l-isomers of methamphetamine and/or amphetamine in biological samples is provided. In some examples, the method includes mixing of a biological sample with an internal standard and diluting with a mobile phase, followed by eluting on a chiral stationary phase contained in a liquid chromatography column. The eluent obtained can then be analyzed using a mass analyzer for the presence of the analytes.

### BACKGROUND

Drug abuse cases have been increasing in number and causing a serious social problem in recent years. Illicit drug abuse is not only harmful to individuals' health but also to the security of society. Many prescription drugs and illicit drugs exist as enantiomers. The two compounds of an enantiomeric pair have identical physical and chemical properties, but differ in the way they react with other chiral compounds as well as the direction in which they rotate plane-polarized light. Since they have identical physical and chemical properties, enantiomeric pairs cannot be resolved by conventional chromatographic methodologies.

Methamphetamine is one such drug which is available in d- and l-forms. The l-enantiomer of methamphetamine, because of its weak stimulating effect on central nervous system, is used in OTC medicines for alleviating symptoms of common colds. The d-enantiomer of methamphetamine, due to its strong stimulating effect on central nervous system, is used as a prescription drug to treat attention deficit hyperactivity disorder and obesity. Since it increases alertness, concentration and energy, it is very attractive to potential abusers.

The l-enantiomers of methamphetamine and amphetamine can be found in urine samples from the subjects who have taken certain OTC medicines with regulatory approvals in European countries and the USA. According to the Department of Health and Human Services (HHS) guidelines, the presence of greater than 20% d-methamphetamine indicates a source other than an OTC product such as DEA schedule II CNS stimulant (d-methamphetamine). Thus, it is crucial that only the assays capable of differentiating among the enantiomers of methamphetamine or amphetamine be employed to identify with accuracy the abuse cases of d-methamphetamine. Enantiomeric analysis helps in determining the percentages of d- and l-forms, which in turn provides the information such as the legal or illegal form of methamphetamine.

In recent years, several approaches have been developed in an attempt to differentiate among the enantiomers of methamphetamine and amphetamine. Most of the approaches involve separation of enantiomers using chiral chromatography. In one approach of chiral chromatography test samples are reacted with a chiral reagent to form diastereomeric salts of methamphetamine or amphetamine, and the diastereomers are then separated using an achiral stationary phase column(s). In such cases, the employed chiral reagent must be present in a highly pure form. Otherwise, undesired diastereomers would form. This makes it more difficult to differentiate diastereomers in a chromatographic analysis using an achiral stationary phase column(s), since both kinds of diastereomers have the same profiles in such a chromatographic analysis. Also the derivation reactions that have to be conducted before analyses not only cause an increase of expenditure of reagents and time, but also a reduction of sensitivity of detection.

Another approach of chiral chromatography includes protocols where test substances are extracted in a liquid or solid phase and concentrated, followed by separation using a chiral stationary phase column. One such method is disclosed in US Publication No. 2013/177994 wherein chiral LC-MS analysis of methamphetamine in urine sample was carried out using macrocyclic glycopeptide chiral columns (ASTEC CHIROBIOTIC™ V2 column). Similar methods involving use of cyclodextrin derivatives chiral stationary phases have been developed (see J Chromatogr B Biomed Appl. 1996; 676(1):35-43 and J Chromatogr B Biomed Appl. 1999; 729(1-2):97-101). In these methods, sample for chromatography is prepared by solid phase extraction method using solid phase extraction cartridge. This method for sample preparation is time consuming as steps like sample pretreatment, conditioning of cartridges, sample loading, washing the cartridge, elution of drugs from cartridge need to be performed. Also it is not cost effective as it involves higher consumption of reagents and solvents for sample preparation.

Thus, there remains a need in the art for simple, rapid and reliable method for the qualitative and quantitative determination of the d- and/or l-enantiomers of methamphetamine and/or amphetamine in bodily fluids and tissues. The subject matter disclosed herein addresses these needs.

### SUMMARY

This summary is intended to introduce, in simplified form, a selection of concepts that are further described in the detailed description. This summary is not intended to identify key or essential features of the claimed subject matter, nor is it intended to be used as an aid in determining the scope of the claimed subject matter.

The disclosed subject matter relates to a method for routine high throughput analysis with accurate results to determine the concentration of d- and/or l-enantiomers of methamphetamine and/or amphetamine in biological samples. For example, the disclosed subject matter relates to a method for determining the concentration of d- and/or l-enantiomers of methamphetamine and/or amphetamine in biological samples by subjecting the sample to high performance liquid chromatography and performing mass spectrometry analysis to detect presence of said enantiomers, wherein sample preparation includes only the addition of an isotope-marked internal standard in to the biological sample followed by dilution with the mobile phase. In the disclosed methods, derivatization or extraction of the analytes by solid phase extraction is not required. The disclosed subject matter can also avoid disadvantages of conventional processes for determining the concentration of d- and/or l-enantiomers of methamphetamine and/or amphetamine in biological samples with liquid chromatography.

In other examples, the disclosed methods include a dilute and shoot method for detection and/or quantification of d- and/or l-enantiomers of methamphetamine and/or amphetamine in the sample, comprising:
a) providing a column comprising a macrocyclic glycopeptide chiral material associated therewith;
b) passing the sample over said column; and
c) performing high performance liquid chromatography tandem mass spectrometry (HPLC/MS/MS) analysis on said sample.

The disclosed methods can include only minimum sample preparation. Labor-intensive, expensive, and somewhat unreliable sample preparation methods like solid phase extraction method and derivatization of the analytes can thus be avoided.

Additional advantages of the disclosed compositions and methods will be set forth in part in the description which follows, and in part will be obvious from the description. The advantages of the disclosed compositions will be realized and attained by means of the elements and combinations particularly pointed out in the appended claims. It is to be understood that both the foregoing general description and the following detailed description are exemplary and explanatory only and are not restrictive of the disclosed compositions, as claimed.

### BRIEF DESCRIPTION OF THE DRAWINGS

The accompanying Figures, which are incorporated in and constitute a part of this specification, illustrate several aspects described below.

FIG. 1 shows the structures of d- and l-enantiomers of methamphetamine and amphetamine;

FIG. 2 is a chromatogram for the calibrator containing d- and l-enantiomers of amphetamine and its internal standard;

FIG. 3 is a chromatogram for the calibrator containing d- and l-enantiomers of methamphetamine and its internal standard;

FIG. 4 is a chromatogram for blank urine, blank urine+IS containing methamphetamine D5, and calibrator containing d- and l-enantiomers of methamphetamine and methamphetamine D5;

FIG. 5 is a chromatogram for calibrator containing d- and l-enantiomers of methamphetamine and methamphetamine D5, blank urine sample spiked with d-enantiomer of methamphetamine and methamphetamine D5, and blank urine sample spiked with 1-enantiomer of methamphetamine and methamphetamine D5

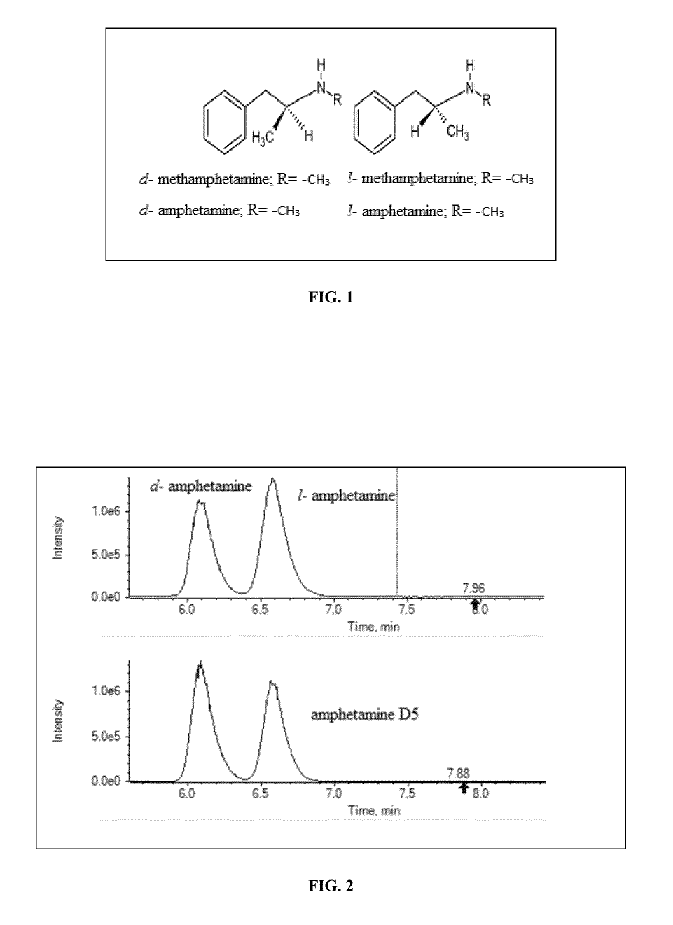

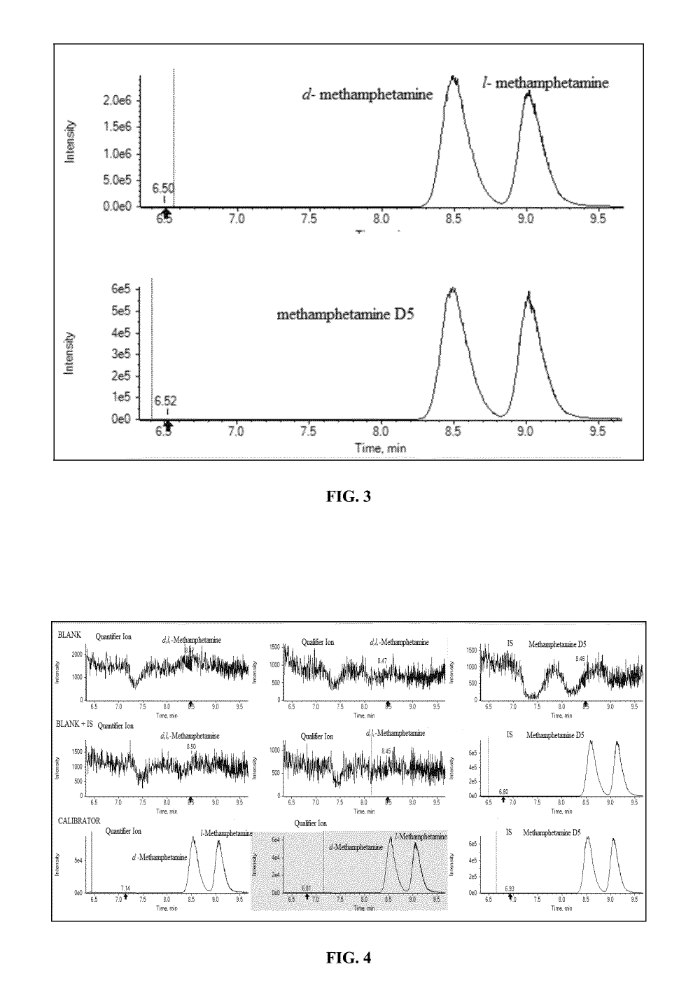

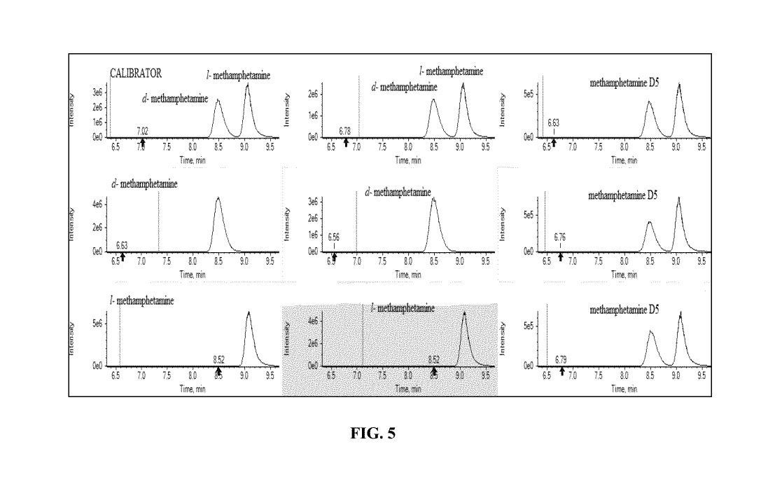

### DETAILED DESCRIPTION

The materials and methods described herein can be understood more readily by reference to the following detailed description of specific aspects of the disclosed subject matter and the Examples and Figures included therein.

Before the present materials and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Definitions

The term “accuracy” is art-recognized and describes the degree of conformity of a measure, i.e., the quantity, to a standard or a true value. For example, an increase in the accuracy of analyte quantification refers to an improvement in obtaining a measured value that is closer to the actual or true value. This improvement may be identified/described by reference to a percent increase in accuracy with respect to the accuracy obtainable using existing methods of measurement.

The term “analyte” refers to any chemical or biological compound or substance that is subject to the analysis of the disclosure. Analytes can include, but are not limited to, small organic compounds, amino acids, peptides, polypeptides, proteins, nucleic acids, polynucleotides, biomarkers, synthetic or natural polymers, or any combination or mixture thereof.

The term “analyzing” or “analysis” refers to a method by which the quantity of each of the individual analytes described herein is detected. Such analysis may be made using any technique that distinguishes between the analyte (or analyte derivative) and the analyte standard (or analyte derivative standard). In some embodiments, the analysis or act of analyzing can include liquid chromatography-tandem mass spectrometry (LC-MS-MS).

The term “chromatographic separation” is art-recognized, and describes a process in which a chemical mixture carried by a liquid or gas is separated into components as a result of differential distribution of the solutes as they flow around or over a stationary liquid or solid phase. For example, chromatographic separations suitable for use in this disclosure can include, but are not limited to liquid chromatographic (including HPLC) methods such as normal-phase HPLC, RP-HPLC, HILIC, and size-exclusion chromatography (SEC), including gel permeation chromatography (GPC). Other suitable methods include additional HPLC methods and related liquid chromatographic techniques, including, e.g., ultra-performance liquid chromatography (HPLC), fast performance liquid chromatography (FPLC) and the like.

The term “internal standard,” refers to a collection of one or more functionalized chemical or biological compounds or substances, e.g., one or more analytes functionalized with another moiety in order to convert such compounds or substances into a derivative thereof. The internal standards are present in known concentrations and added to the sample to form a sample mixture. The addition of the internal standard allows for the detection of and comparison between the known concentrations of one or more known analytes, with the unknown concentrations of analytes in the original sample. As such, the internal standards can provide a way to measure the absolute quantity of an analyte in sample using a response factor calculation.

The term “liquid chromatography” is art-recognized and includes chromatographic methods in which compounds are partitioned between a liquid mobile phase and a solid stationary phase. Liquid chromatographic methods are used for analysis or purification of compounds. The liquid mobile phase can have a constant composition throughout the procedure (an isocratic method), or the composition of the mobile phase can be changed during elution (e.g., a gradual change in mobile phase composition such as a gradient elution method).

The term “mass spectrometry” and “mass spectroscopy” are art-recognized and used interchangeably to describe an instrumental method for identifying the chemical constitution of a substance by means of the separation of gaseous ions according to their differing mass and charge. A variety of mass spectrometry systems can be employed to analyze the analyte molecules of a sample subjected to the disclosed methods. For example, mass analyzers with high mass accuracy, high sensitivity and high resolution may be used and include, but are not limited to, atmospheric chemical ionization (APCI), chemical ionization (CI), electron impact (EI), fast atom bombardment (FAB), field desorption/field ionization (FD/FI), electrospray ionization (ESI), thermospray ionization (TSP), matrix-assisted laser desorption (MALDI), matrix-assisted laser desorption time-of-flight (MALDI-TOF) mass spectrometers, ESI-TOF mass spectrometers, and Fourier transform ion cyclotron mass analyzers (FT-ICR-MS). In addition, it should be understood that any combination of MS methods could be used in the methods described herein to analyze an analyte in a sample. In certain embodiments, the MS technique used for analysis of the analyte described herein is one that is applicable to most polar compounds, including amino acids, e.g., ESI.

The term “mobile phase” is art-recognized, and describes a solvent system (such as a liquid) used to carry a compound of interest into contact with a solid phase (e.g., a solid phase in a solid phase extraction (SPE) cartridge or HPLC column) and to elute a compound of interest from the solid phase.

The term “precision” is art-recognized and describes the reproducibility of a result. It is measured by comparison of successive values obtained for a measurement to the prior values, where more precise measurements (or those with greater precision) will be demonstrated by successive measurements that are more consistently closer to the prior measurements.

The terms “quantitative” and “quantitatively” are art-recognized and refers to measurements of quantity or amount. For example, the term “quantification” describes the act of measuring the quantity or amount of a particular object, e.g., an analyte. In some embodiments, the quantitative analysis can be a measurement of an absolute amount, as opposed to relative amount, i.e., the total amount of analyte may be quantified absolutely in order to determine the actual amount of the analyte.

The term “sample” refers to a representative portion of a larger whole or group of components that are capable of being separated and detected by the disclosed methods. Exemplary samples include chemically or biologically derived substances, e.g., analytes of the disclosed methods. In particular embodiments, the components of the sample include, but are not limited to small organic compounds, amino acids, peptides, polypeptides, proteins, nucleic acids, polynucleotides, biomarkers, synthetic or natural polymers, or any combination or mixture thereof.

The term “sample mixture,” refers to the resultant product when a sample is mixed or combined with one or more analyte derivative standards, e.g., of a known concentration.

The disclosed subject matter relates to methods for routine high throughput analysis with accurate results to determine the concentration of d- and/or l-isomers of methamphetamine and/or amphetamine in biological samples.

In one embodiment, disclosed herein are methods of detecting d- and/or l-enantiomers of methamphetamine and/or amphetamine in a biological sample from a subject, comprising:
(a) preparing a sample for liquid chromatography by adding an internal standard into the biological sample;
(b) subjecting the sample to liquid chromatography column comprising a chiral stationary phase; and
(c) analyzing the eluent for the presence of said enantiomers using a mass analyzer.

In one embodiment, disclosed herein are methods of determining the concentration of d- and l-enantiomers of methamphetamine and amphetamine in biological samples using a dilute and shoot chiral chromatography mass spectrometry method.

The sample preparation in the disclosed methods for the quantitative analysis of d- and/or l-isomers of amphetamine or methamphetamine can include two steps, which can be performed quickly and easily.

In the first step, the sample (blood plasma, urine, or cell culture medium) is mixed with a solution of the internal standards. In one embodiment, deuterated internal standards like amphetamine-d₅ and methamphetamine-d₅ (commercially available) are added to the sample.

The mixture obtained after addition of internal standards to the sample can be subjected to centrifugation. In one embodiment, the sample is centrifuged between 10,000 RPM to 15,000 RPM for 15 to 25 minutes. Preferably, the sample is centrifuged at 14,000 RPM for 20 minutes.

In the second step, conventional HPLC mobile phase is added to the sample. In one of the embodiment the conventional HPLC mobile phase is a mixture of methanol/water/ammonium trifluoroacetate. The % w/v ammonium trifluoroacetate can have values from 0.02 to 0.1%, the v/v ratio of methanol:water can have values from 2:98 to 10:90. Preferably, the 0.05% w/v Ammonium trifluoroacetate in water:methanol (2:98, v/v).

The sample prepared in this manner is directly subjected to HPLC separation. The volume of the sample that is subjected to HPLC separation can range from 2 μL to 10 μL. Preferably, the volume of the sample that is subjected to HPLC separation is 5 μL.

In some embodiments, the chiral analytical column can be normal phase or reverse phase. In another embodiment the column is reverse phase comprising macrocyclic glycopeptides, cyclodextrin derivatives or suitable antibiotic like vancomycin. Preferred stationary phase comprises macrocylic glycopeptides. The flow rate in the column can range from 0.7 mL/min to 1 mL/min. Preferably, the flow rate in the column is 0.75 mL/min.

The eluent obtained after HPLC can be ionized using suitable techniques like electrospray ionization, thermal ionization, gas ionization and like. Preferably, the ionization technique is electrospray ionization.

The analytes can be detected by any suitable mass analyzer like quadrupole mass spectrometer, ion trap mass spectrometer, time of flight mass spectrometer. Preferably, quadrupole mass analyzer is used for detection of analytes.

Concentration of d- and/or l-enantiomers of methamphetamine and/or amphetamine can be calculated based on area counts of the drug relative to area counts of analogous internal standard ions.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

### Instrument and Equipment

Mass Spectrometer: ABSciex 4500 Triple Quadrupole Mass Spectrometer

Pumps: Shimadzu LC system

Chromatography Column: Astec Chirobiotic V2, (25 cm×4.6 mm×5 μm)

Multiple Reaction Monitoring (MRM) was carried out to detect the analytes.

Parameters such as Declustering Potential (DP), Entrance Potential (EP), Collision Cell Entrance Potential (CEP), Collision Cell exit potential (CXP) were optimized. All these voltages were optimized for each analyte in the assay by compound optimization and the values were incorporated into the acquisition method.

### Reagents

Internal Standards—Deuterated internal standards (IS) were purchased from Cerilliant, Inc. and were diluted to appropriate concentrations from which a stock IS solution was prepared. Working IS was a mixture of Amphetamine D5 and Methamphetamine D5 of concentration 1000 ng/mL in urine.

Reference Standards—d,l-methamphetamine, l-methamphetamine, d-methamphetamine, d,l-methamphetamine-d₅, d,l-amphetamine, l-amphetamine, d-amphetamine, d,l-amphetamine-d₅ were purchased from Cerilliant chemicals (Round Rock, Tex., USA).

Solvents—Optima grade methanol was purchased from Fisher Scientific (Fair Lawn, N.J., USA). Reverse osmosis, as well as distilled and deionized water from Myron was used.

Salt—Ammonium trifluoroacetate (ATA) was purchased from Sigma Aldrich.

Calibrators—Stock Calibrators were diluted to appropriate concentrations. A standard stock solution was prepared with all the analytes of interest in methanol and stored in the freezer. Five levels of working standards were prepared from the stock solution. Stock solution, working standards in aliquots were stored as per manufacturers' recommendations, and were stable until manufacturers' listed expiration date. All the calibrators and controls were prepared in the blank urine.

### Quality Controls

Following controls were run with each batch:

Negative control is purchased from Utak Laboratories Inc.

Positive Threshold control (In House control), D80, and D20 controls are prepared from Cerilliant standards.

d-mix with d-Amphetamine and d-Methamphetamine, l-mix with l-Amphetamine and l-Methamphetamine is prepared to identify the single enantiomers.

TABLE I
Concentrations of calibrators and controls in ng/mL

| | Cal- | Cal- | Cal- | Cal- | Cal- | In- | | |
|---|---|---|---|---|---|---|---|---|
| Analyte | 1 | 2 | 3 | 4 | 5 | house | D80 | D20 |
| d-Amphetamine | 50 | 250 | 500 | 1250 | 2500 | 500 | 800 | 200 |
| l-Amphetamine | 50 | 250 | 500 | 1250 | 2500 | 500 | 200 | 800 |
| d- | 50 | 250 | 500 | 1250 | 2500 | 500 | 800 | 200 |
| Methamphetamine | | | | | | | | |
| l- | 50 | 250 | 500 | 1250 | 2500 | 500 | 200 | 800 |
| Methamphetamine | | | | | | | | |

### Purification and Extraction Procedure

The working solutions (calibrators, controls, and internal standards) were allowed to come to the room temperature. 100 μL of calibrators, controls and specimens were added into the labeled bullet tubes followed by addition of 100 μL of internal standard. The bullet tubes were capped and vortex mixed followed by centrifugation at 14,000 RPM for 20 minutes.

To 1.0 mL labeled vials 500 μL of mobile phase was added. 100 μL of supernatant from the bullet tube was transferred into 1.0 mL vial with mobile phase in it. The vials were capped and the samples were vortex mixed.

The MS-MS parameters used for quantification of amphetamine and methamphetamine are shown in Table II below.

TABLE II

| Q1 Mass | Q3 Mass | Time | | | | | |
|---|---|---|---|---|---|---|---|
| (Da) | (Da) | (Mins) | ID | DP | EP | CE | CXP |
| 136.115 | 119 | 7.28 | L-Amphetamine | 60 | 10 | 11 | 8 |
| 136.141 | 91 | 7.28 | D-Amphetamine | 64 | 10 | 21 | 10 |
| 150.134 | 91 | 8 | D-Methamphetamine | 53 | 10 | 39 | 8 |
| 150.094 | 119 | 8 | L-Methamphetamine | 53 | 10 | 15 | 8 |
| 155.35 | 121 | 8 | Methamphetamine-D5 | 26 | 10 | 15 | 10 |
| 141.176 | 124 | 7.28 | Amphetamine-D5 | 41 | 10 | 13 | 6 |

Q1—Quadrapole one,
Q3—Quadrapole three,
DP—declustering potentials,
EP—entrance potentials,
CE—collision energies,
CXP—collision cell exit potentials.

### Loading the Curve and a Batch of Samples

Curve was loaded in the following order:

Blank, Blank+IS, Cal-1, Cal-2, Cal-3, Cal-4, Cal-5, In-House, and BL1 followed by the samples. Activate the corresponding acquisition method. Use the corresponding quantitation method.

Liquid chromatography and mass spectrometry parameters used for quantification of amphetamine and methamphetamine are shown in Table III and Table IV below.

TABLE III
Chromatography details

| | |
|---|---|
| Chromatography mode | Reverse phase |
| Isocratic/gradient method | Isocratic |
| Mobile Phase | 0.05% w/v Ammonium |
| | trifluoroacetate in water:methanol |
| | (2:98, v/v) |
| Needle Wash | 40.0% Isopropanol + 40.0% |
| | Acetonitrile + 20.0% Acetone |
| Flow rate | 0.75 mL/min |
| Run time | 10 minutes |
| Sample injection volume | 5 μL |
| Column temperature | 32° C. |

TABLE IV
Ion source parameters

| Interface | Electrospray ionization | |
|---|---|---|
| Ionization mode | Positive | |
| Source/Gas temperature | 600° | C. |
| Ion source gas 1 | 50 | PSI |
| Ion source gas 2 | 50 | PSI |

What is claimed is:

1. A method of detecting d- and/or l-enantiomers of methamphetamine and/or amphetamine in a biological sample from a subject, comprising:
(a) preparing a sample for liquid chromatography by adding an internal standard into the biological sample;
(b) subjecting the sample to liquid chromatography column comprising a chiral stationary phase; and
(c) analyzing the eluent for the presence of said enantiomers using a mass analyzer.

2. The method of claim 1, further comprising centrifuging the sample after adding the internal standard.

3. The method of claim 2, further comprising diluting the sample after centrifuging the sample.

4. The method of claim 1, wherein the chiral stationary phase comprises macrocyclic glycopeptides.

5. The method of claim 1, wherein the biological sample is a bodily fluid selected from the group consisting of saliva, sweat, urine, blood, serum, plasma, spinal fluid, and combinations thereof.

6. The method of claim 1, wherein the eluent is ionized prior to analyzing.

7. The method of claim 6, wherein the eluent is ionized via electrospray ionization technique.

8. The method of claim 1, wherein the mass analyzer is a quadrupole mass spectrometer.

9. The method of claim 1, wherein derivatization or extraction of the enantiomer by solid phase extraction is not performed.
